(19) United States
(12) Patent Application Publication
(10) Pub. No.: US 2020/0299355 A1
(43) Pub. Date: Sep. 24, 2020

(54) METHODS FOR DIAGNOSING AND TREATING DISEASES BASED ON MODULATING DRUG EFFLUX BY BINDING TO CRYPTIC REGION OF CD44

(71) Applicant: Splash Pharmaceuticals, Inc., San Diego, CA (US)

(72) Inventors: Malcolm Finlayson, San Diego, CA (US); David Nelson, San Diego, CA (US)

(21) Appl. No.: 16/894,439

(22) Filed: Jun. 5, 2020

Related U.S. Application Data
(63) Continuation-in-part of application No. 16/072,134, filed on Jul. 23, 2018.
(60) Provisional application No. 62/858,781, filed on Jun. 7, 2019.
(60) Provisional application No. 62/290,306, filed on Feb. 2, 2016.
(60) Provisional application No. 62/314,867, filed on Mar. 29, 2016.
(60) Provisional application No. 62/368,964, filed on Jul. 29, 2016.

(51) Int. Cl. C07K 14/705

(52) U.S. Cl. CPC C07K 14/70585; A61K 45/06

(57) ABSTRACT

The present invention relates to methods for modulating efflux of drugs based on binding to a protein region of CD44 in part comprising the amino acid sequence specified in the provisional application referenced above, and incorporated in its entirety by reference herein (“PROV”). In some aspects, the present invention relates to methods for treatment of cancer by preventing the cellular efflux of therapeutic drugs; for example, preventing the cellular efflux of anti-cancer agents, for example a taxane, by contacting a CD44-modulating peptide, comprising the amino acid sequence specified in the provisional application referenced above, and incorporated in its entirety by reference herein (“PROV”).

### CROSS-REFERENCES TO RELATED APPLICATIONS

This application is a non-provisional and claims benefit of U.S. Provisional Patent Application No. 62/858,781, filed Jun. 7, 2019, the specification(s) of which is/are incorporated herein in their entirety by reference.

This application is a continuation-in-part and claims benefit of U.S. patent application Ser. No. 16/072,134, filed Jul. 23, 2018, which is a 371 application and claims benefit of PCT/US17/15754, filed Jan. 31, 2017, which claims benefit of U.S. Provisional Patent Application Nos. 62/290,306, filed Feb. 2, 2016, U.S. Provisional Patent Application No. 62/314,867, filed Mar. 29, 2016, and U.S. Provisional Patent Application No. 62/368,964, filed Jul. 29, 2016, the specification(s) of which is/are incorporated herein in their entirety by reference.

### BACKGROUND OF THE INVENTION

### Field of the Invention

The present invention relates to methods for treating diseases by modulating efflux of drugs based on binding to a protein region of CD44 in part comprising the amino acid sequence specified in the provisional application referenced above, and incorporated in its entirety by reference herein (“PROV”) In some aspects, the present invention relates to methods for treatment of cancer by preventing the cellular efflux of therapeutic drugs; for example, preventing the cellular efflux of anti-cancer agents, for example a taxane, by contacting or binding a CD44-modulating peptide.

### Background Art

Mortality due to cancer is generally the result of metastasis of the primary tumor. Recurrence at local and/or distant sites following first-line therapy continues to be a major challenge. As such, strategies are needed that more effectively treat the tumor and inhibit the metastatic process in cancer patients including drugs or methods that enhance efficacy of, reduce resistance to, and/or prevent cellular efflux of anti-cancer agents. Provided herein are solutions to these problems and other problems in the art.

The metastatic process involves migration and invasion of tumor cells from the local microenvironment, intravasation into the blood or lymph circulation, extravasation from circulation back into tissue, followed by metastatic colonization and growth or dormancy. Metastasis and recurrence have been linked to a subpopulation of highly invasive tumorigenic cells, which have been shown to be resistant to chemotherapeutics. These tumorigenic cells are characterized by the expression of CD44, a multifunctional receptor involved in cell signaling, adhesion, migration, and proliferation. CD44 functions as a receptor, as a co-receptor (e.g., c-Met and EGFR), and as a platform for MMPs to enable many biological processes. In addition, CD44 is known to mediate invasion and metastasis.

Chemotherapeutic resistance has been linked to a number of CD44 pathways including multi-drug resistant (MDR1)-dependent efflux of chemotherapeutics. This resistance results in expansion of invasive cells following first-line chemotherapy, which leads to recurrence. Studies have shown that targeting CD44 or related signaling pathways, using RNAi strategies or with anti-CD44 antibodies, suppress tumor growth and relapse, and increase sensitivity of these cells to chemotherapeutics. This supports the utility of the present invention in that targeting CD44 may render tumor cells more sensitive to therapeutic agents. Without limiting the invention to a particular theory or mechanism, this synergy may result from inhibiting CD44-regulated drug efflux mechanisms.

Efflux pump inhibitors prevent the energy-dependent efflux of drugs and some endogenous metabolites from the cells. They are a promising strategy for restoring the activity of existing antimicrobial and cytotoxic agents. A majority of the efflux pump inhibitors are not used as pump inhibitors in routine clinical practice because concentrations that achieve efflux inhibition in vitro are rarely achieved in vivo without serious toxicities. At present, many are used only for epidemiological surveys of drug-resistant organisms.

CD44 is a complex multifunctional receptor modulating a variety of cellular processes. Studies described herein demonstrate that a peptide comprising amino acid sequence specified in the PROV inhibits the metastatic process in a CD44 dependent manner. Previous findings showed that A6 does not produce a global nonspecific change in CD44, but instead produces a subtle change to a specific epitope, for example to an epitope according to amino acid sequence specified in the PROV Because CD44 is associated with a chemo-resistant and radiation-resistant phenotype, which is countered by inhibition of CD44 signaling, the use of A6 may inhibit CD44-mediated resistance. A6 may be used in combination with a cytotoxic chemotherapeutic agent or radiation therapy to increase cytotoxicity of chemotherapeutic agents or radiation to inhibit metastases and to render resistant cells sensitive to chemotherapy. Furthermore, due to the positive safety profile documented for A6, there would be a reduced likelihood of compounding toxicity. As such, A6 may be combined with almost any chemotherapeutic or radiation therapy, or a combination of both.

This safety profile also invites the use of A6 for longer-term maintenance therapy to prevent recurrence stemming from micrometastases surviving first-line standard of care treatment. A6 has demonstrated activity against CD44 expressing tumor cells and CLL cells, and is a candidate for the treatment of malignant disease and hematological malignancy. A6 has demonstrated clinical safety and efficacy, and by targeting CD44 resistant cells to prevent metastases and recurrence, has the possibility of creating a new paradigm for cancer treatment.

Preclinical studies have shown that A6 has anti-migratory, anti-invasive, and anti-metastatic properties. A6 inhibits migration and invasion of breast, lung, glioma, ovarian, and prostate cancer cell lines in vitro in a dose-dependent manner, and inhibits the growth and metastasis of breast, melanoma, glioma, lung, and prostate cancer cells in xenograft models in vivo. The combination of A6 with tamoxifen resulted in an inhibition of breast tumor cell growth greater than with either A6 or tamoxifen alone. A similar result was observed in glioma xenograft studies where the combination of A6 with cisplatin also inhibited tumor cell growth greater than with either A6 or cisplatin alone.

Studies also have been conducted to evaluate the safety and efficacy of A6. These include safety studies in healthy volunteers as well as studies in patients with varying stages of metastatic disease.

Normal Volunteers:

A6 was administered to normal volunteers in a Phase 1a, double-blind, placebo-controlled, parallel-group clinical trial. Results showed there were no systemic drug-related adverse events. No significant alterations in physical examinations, vital signs, electrocardiograms or clinical laboratory testing, including coagulation parameters such as PT, PTT, fibrinogen, and thrombin time, were noted. Pharmacokinetic data in normal volunteers at the 150 mg/day and 300 mg/day single dose levels showed a t_{1/2} of 1.8-2.0 hours at both dose levels. Furthermore, no cumulative increase in concentration over time was detected. Following A6 subcutaneous administration twice daily for 6 days, no anti-A6 antibody production was detected at day 14.

Advanced Gynecologic Cancer:

A Phase 1 b trial, was conducted in women with advanced gynecologic cancer. Greater than 40% of patients dosed continuously with A6 experienced disease stabilization. The study used a sequential dose-escalation design, with the lowest-dose group (4 patients) receiving A6 for cycles of 14 days “on” followed by 14 days “off”, a regimen not expected to produce any therapeutic effect. Twelve patients with advanced gynecologic malignancies that had failed standard therapy were treated with daily, uninterrupted A6. In this population, in which disease progression is expected, 5 patients (4 of whom had ovarian or primary peritoneal carcinoma) achieved stable tumor measurements for at least 4 months, and 1 for greater than 12 months. Patients continued treatment until disease progression or unacceptable toxicity. Response was evaluated as defined by RECIST and the Gynecologic Cancer Intergroup (GCIG) CA-125 response criteria. A Kaplan-Meier retrospective analysis demonstrated that patients treated with daily A6 showed a delayed time to tumor progression relative to an effective control group, (whose treatment was intermittent and, therefore, not expected to have beneficial effect) providing evidence of antineoplastic activity. Continuous treatment with A6 resulted in an increased time to progression (TTP) with a median TTP of 78 days (95% CI 57, 365) compared to 44 days (95% CI 4, 62) in patients who received the intermittent therapy (log-rank p-value=0.02). The safety outcome in this Phase 1 b gynecologic cancer trial was excellent and showed no specific toxicity profile.

Asymptomatic Progression of Ovarian Cancer:

A randomized, double-blind, placebo-controlled Phase 2 clinical trial evaluating A6 in women with asymptomatic CA-125 progression of ovarian cancer (“marker-only relapse” or MOR) was conducted. Patients were in clinical remission after first-line chemotherapy with no evidence of disease following physical examination or imaging analysis, but had two consecutive, above-normal, increases of CA125 (a biomarker for recurrence/poor prognosis). Because patients were clinically asymptomatic at the time of entry, the study was able to be placebo-controlled. The primary endpoints were time to clinical progression of disease and safety of A6. The secondary endpoints included changes in serum CA125. This study enrolled 24 patients: 12 were randomized to daily self-administration of A6 at two doses, and 12 to matching placebo injections. Both groups were followed for up to 9 months. Although there were no complete responses, 36% of patients achieved stable disease. A6 treatment was not associated with CA125 response. Results from a Kaplan-Meier analysis of progression-free survival showed that treatment with A6 significantly prolonged time to progression. Despite the small patient sample size, A6 therapy was associated with a statistically significant increase in progression free survival (PFS) (log-rank p-value=0.01) with a median PFS of 100 days (95% CI 64, 168) compared to 49 days (95% CI 29,67) in patients who received the placebo. Furthermore, the safety profile of A6 was comparable to that of control (placebo) treatment.

Persistent or Recurrent Ovarian Cancer:

A Phase 2 trial was conducted in patients with persistent or recurrent epithelial ovarian, fallopian tube, or primary peritoneal carcinoma to evaluate A6 in a patient population with a disease burden greater than that presented in the previously described MOR trial. Patients had received one prior platinum-based chemotherapeutic regimen and were allowed to have received one additional cytotoxic regimen for the management of recurrent or persistent disease. Patients received a 150 mg twice daily subcutaneous dose of A6 and continued on treatment until disease progression or unacceptable toxicity. Response criteria were as defined by RECIST. Primary measures of clinical efficacy were objective tumor response and PFS at 6 months compared to a historical Gynecologic Oncology Group (GOG) dataset based on a similar population of patients. Of the 31 eligible patients evaluated, no responses were observed; 6.5% were progression free for at least 6 months; and 36% of evaluable patients achieved stable disease. A6 was well tolerated but had minimal activity in patients with persistent or recurrent epithelial ovarian, fallopian tube, or primary peritoneal carcinoma under the conditions of this trial.

### BRIEF SUMMARY OF THE INVENTION

It is an objective of the present invention to provide methods that allow for preventing cellular efflux of a therapeutic agent that has been internalized into a cell based on an A6 peptide (or a CD44-modulating peptide or A6 peptidomimetic) binding to a region of a protein associated with a CD44-expressing cell. The region of the protein in part may comprise amino acid sequence, specified in the PROV, or a glycosylated amino acid sequence specified in the PROV. Embodiments of the invention are given in the dependent claims. Embodiments of the present invention can be freely combined with each other if they are not mutually exclusive.

The present invention also features a method for preventing efflux of an anti-cancer agent that has been internalized into a CD44 expressing cell. The method comprises administering the anti-cancer agent to the cell, that contains the anti-cancer agent and has mechanisms for drug efflux and contacting or binding a peptide to a cryptic region of a protein in a CD44 expressing cell. The cryptic region of a protein in part may comprise amino acid sequence specified in the PROV, occupying amino acid positions 120-127 of CD44 or a glycosylated amino acid sequence specified in the PROV, and wherein said contacting of peptide to SEQ ID NO:3 prevents the efflux of the anti-cancer-agent from the cell.

The present invention further features a method of inhibiting the growth and migration of a CD44 expressing tumor. The method comprises: (a) identifying tumors with CD44 expression; (b) administering an anti-cancer agent (e.g., a taxane) to the CD44 expressing tumor; and (c) administering an A6 peptide that binds to a cryptic region of CD44, wherein the cryptic region in part comprises amino acid sequence specified in the PROV, and wherein the A6 peptide binding to a sequence specified in the PROV prevents the efflux of the anti-cancer-agent from the cell to inhibit the growth of the tumor.

The present invention also features a method of treating a cancer subject with CD44 expressing tumors, the method comprises: (a) identifying subjects with CD44 expressing tumors; (b) treating the subjects with CD44 expressing tumors with an anti-cancer agent (e.g., a taxane); and (c) administering an A6 peptide that binds to a cryptic region of CD44, wherein the cryptic region in part comprises amino acid sequence specified in the PROV or a glycosylated amino acid sequence specified in the PROV, and wherein the A6 peptide binding to a sequence specified in the PROV prevents the efflux of the anti-cancer agent from the cell to effectively treat the subject.

One of the unique and inventive technical features of the present invention is the binding of an agent (e.g., A6 peptide, or a CD44-modulating peptide) to an amino acid sequence in part comprising a sequence specified in the PROV of a protein in a cell, preventing cellular efflux of the therapeutic agent. For example, the present invention features a method that combines an anti-cancer agent (e.g., a taxane) and a peptide that binds to a sequence specified in the PROV, of which this binding prevents cellular efflux of the taxane, improving the treatment response to the taxane. Without wishing to limit the invention to any theory or mechanism, it is believed that the technical feature of the present invention, binding to a sequence specified in the PROV, advantageously provides for decreasing the efflux and improving the efficacy of therapeutic agents, e.g., taxanes. None of the presently known prior references or work has the unique inventive technical feature of the present invention.

Furthermore, the prior references teach away from the present invention. For example, results from previous pre-clinical studies showed an improvement in the inhibition of tumor growth when A6 was combined with tamoxifen or with cisplatin as compared to either agent alone, but no improvement in the inhibition of tumor growth or survival was observed with the addition of taxane to A6 (FIGS. 3-4). In addition, a majority of the efflux pump inhibitors are not used as pump inhibitors in routine clinical practice because concentrations that achieve efflux inhibition in vitro are rarely achieved in vivo without serious toxicities. Therefore, the prior art discourages the use of combination therapy, particularly with A6 and a taxane for modulating efflux of therapeutic agents.

Furthermore, the inventive technical features of the present invention contributed to a surprising result. For example, in one compassionate use case of a patient with stage 4 endometrial cancer and metastases to the lung, treated with paclitaxel in combination with A6, the physician was surprised to observe that this patient has been in remission for at least 13 years with no sign of disease, even 5 years after stopping A6, because the physician hadn't known of anyone surviving that long after the cancer has spread to the lungs. The other compassionate use case of A6 in combination with paclitaxel in a patient with refractory, triple-negative breast cancer (a breast cancer that is estrogen receptor negative, progesterone receptor negative, and HER2 negative) resulted in the patient being in complete remission with no evidence of distant metastasis or local recurrence of the cancer for at least nine years; A6 was discontinued and the patient is still in clinical remission five years after A6 discontinuation, a surprising result for this patient with triple-negative breast cancer, a very difficult type of breast cancer to treat that is refractory to most anti-cancer agents.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a semi-transparent molecular surface representation of the homology model of hCD44 built with SWISS-MODEL using the structure of mCD44 in complex with HA8 (PDB 2JCR) as template. The HA8 binding pocket is depicted in orange while in purple are highlighted the putative glycosylation sites. The most perturbed residues are labeled and localized in a putative back pocket opposite from the HA8 binding pocket. The red square indicates the portion of hCD44 with sequence homology with A6 peptide.

FIG. 1B shows the cryptic binding site, an unexpected binding site for a peptide on the back side of chain B, A6 binding site (yellow) in relation to the known HA binding site (orange). (HA position modelled by superposition of murine HABD structure 2JCR).

FIG. 2 illustrates that the A6 polypeptide of a sequence specified in the PROV shares sequence homology with a portion of the Link-Domain of CD44, the cryptic binding region, a sequence specified in the PROV.

FIG. 3 illustrates testing of A6 plus paclitaxel (PTX) in the B16F10-DsRed Cell Lung Metastasis Model. Tumor burden (number of tumor nodules) in the lungs was not significantly reduced by A6 in the presence or absence of paclitaxel.

FIG. 4 Illustrates testing of A6 plus paclitaxel (PTX) in HEY [cisplatin (DDP) sensitive] and HEY/C2 (DDP resistant) cells. A6 did not affect sensitivity of HEY or HEY/C2 cells to paclitaxel.

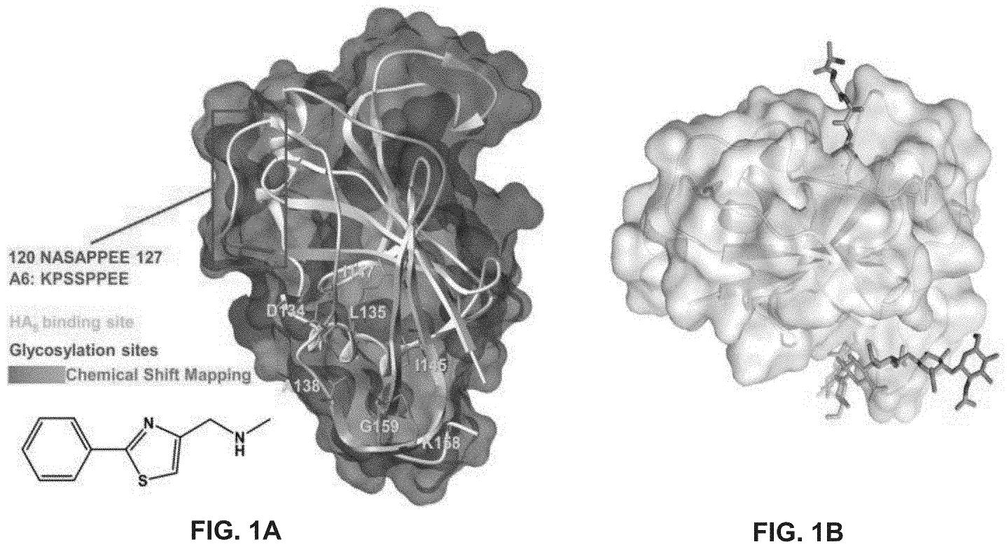

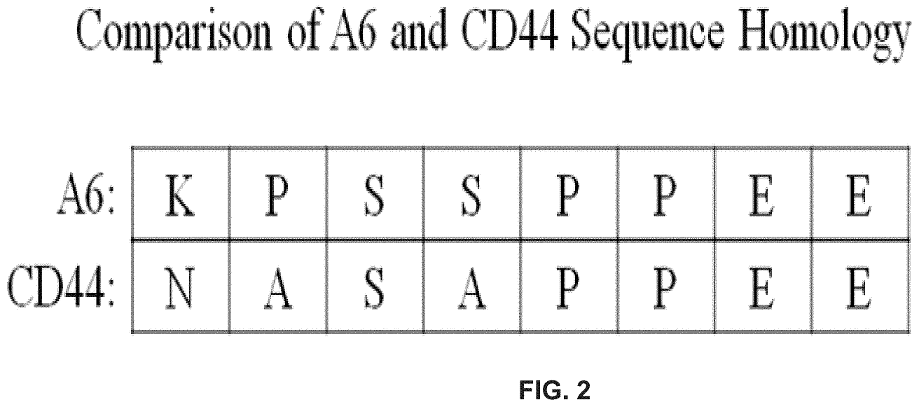

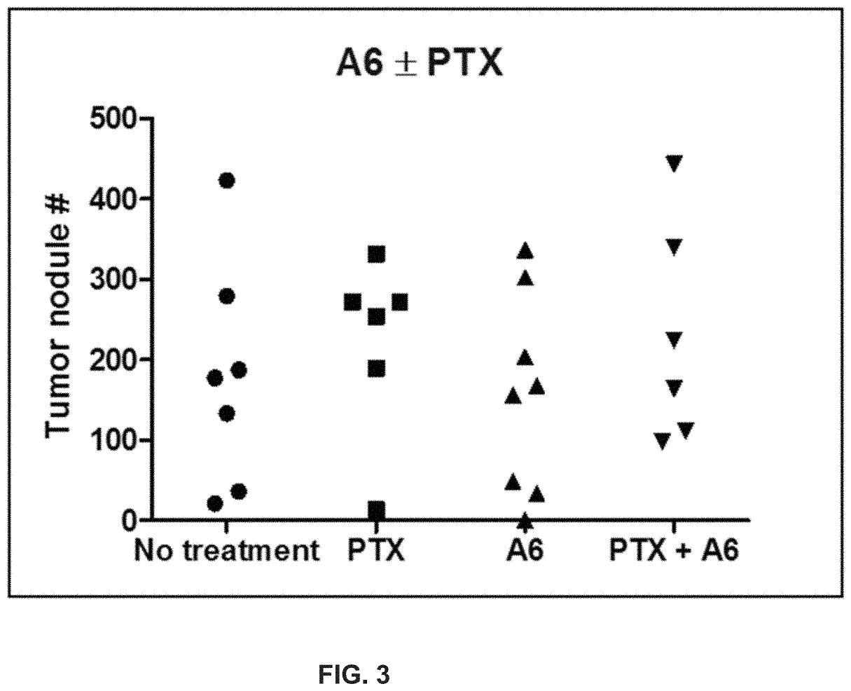

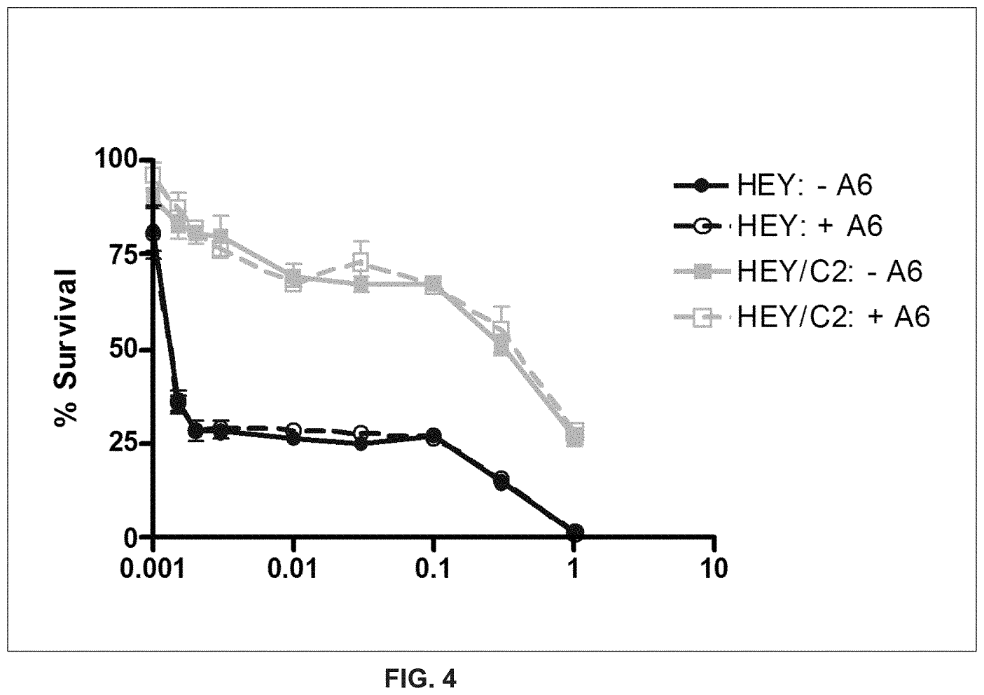

### DETAILED DESCRIPTION OF THE INVENTION

The terms “polypeptide” and “protein” are used interchangeably herein and refer to any molecule that includes at least 2 or more amino acids.

As used herein, “administering” and the like refer to the act physically delivering a composition or other therapy (e.g. a radiation therapy) described herein into a subject by such routes as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. Radiation therapy can be administered using techniques described herein, including for example, external beam radiation or brachytherapy. When a disease, disorder or condition, or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof.

The term “coadministration” refers to administration of two or more agents (e.g., a polypeptide described herein and another active agent such as an anti-cancer agent or other therapy (e.g. a radiation therapy) described herein). The timing of coadministration depends in part on the combination and compositions or other therapies administered and can include administration at the same time, just prior to, or just after the administration of one or more additional therapies, for example cancer therapies such as chemotherapy, hormonal therapy, radiotherapy, or immunotherapy. Coadministration is meant to include simultaneous or sequential administration of a composition or therapy individually or in combination (more than one polypeptide described herein or an anti-cancer agent described herein or radiation therapy as described herein). Coadministration can include administration of two or more agents where the agents are optionally combined with other active substances (e.g., to reduce metabolic degradation). The polypeptides, anti-cancer agents and radiation therapies described herein can be used in combination with one another, with other active agents known to be useful in treating a disease associated with cells expressing a particular kinase as described herein, or with adjunctive agents that cannot be effective alone, but can contribute to the efficacy of the active agent.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances, the term patient refers to a human.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

An improvement in the cancer or cancer-related disease can be characterized as a complete or partial response. Complete response refers to an absence of clinically detectable disease with normalization of any previously abnormal radiographic studies, bone marrow, and cerebrospinal fluid (CSF) or abnormal monoclonal protein measurements. Partial response refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

A refractory, resistant, or persistent cancer refers to a circumstance where patients, even after intensive treatment, have residual cancer cells (e.g., leukemia cells, lymphoma cells, circulating tumor cells or cancer stem cells) in their lymphatic system, blood and/or blood forming tissues (e.g., marrow).

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The term “preventing” refers to the treatment with or administration of a polypeptide or agent (e.g. anti-cancer agent described herein) provided herein, with or without other additional active agent (e.g. an anti-cancer agent), prior to the onset of symptoms, particularly to patients at risk of cancer and/or other disorders described herein. The term also refers to coadministration of a polypeptide with other therapies including radiation therapies as described herein. It should be understood that the polypeptides described herein can be co-administered with one or more anti-cancer agents and radiation therapies described herein. The term prevention includes the inhibition or reduction of a symptom of the particular disease, as well as a reduced incidence of a symptom of the particular disease (e.g. by comparison to historical data for a given subject, or population data for similar subjects). Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

A prophylactically effective amount of a polypeptide or agent (e.g. an anti-cancer agent described herein) means an amount of therapeutic agent, alone or in combination with other agents, which provides a prophylactic benefit in the inhibition or reduced incidence of a symptom of a disease or recurrence of a disease. The term also refers to coadministration of a polypeptide described herein with other therapies including radiation therapies as described herein. The term prophylactically effective amount can encompass an amount that improves overall prophylaxis or enhances the prophylactic efficacy of another prophylactic agent.

The term “effective amount” as used herein refers to the amount of a therapy (e.g., a composition or radiation therapy provided herein) which is sufficient to reduce and/or ameliorate the severity and/or duration of a given disease, disorder or condition and/or a symptom related thereto. This term also encompasses an amount necessary for the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of a polypeptide described herein, an anti-cancer agent described herein, or a radiation therapy described herein is an amount sufficient to provide a therapeutic benefit in the treatment or management of a cancer, or to delay or minimize one or more symptoms associated with the presence of the cancer. A therapeutically effective amount of a polypeptide described herein, an anti-cancer agent described herein, or a radiation therapy described herein means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent.

A therapy is any protocol, method and/or agent that can be used in the prevention, management, treatment and/or amelioration of a given disease, disorder or condition. In certain embodiments, the terms “therapies” and “therapy” refer to a drug therapy, biological therapy, supportive therapy, radiation therapy, and/or other therapies useful in the prevention, management, treatment and/or amelioration of a given disease, disorder or condition known to one of skill in the art such as medical personnel.

A regimen is a protocol for dosing and timing the administration of one or more therapies (e.g., combinations described herein, another active agent such as for example an anti-cancer agent described herein, or a radiation therapy described herein) for treating a disease, disorder, or condition described herein. A regimen can include periods of active administration and periods of rest as known in the art. Active administration periods include administration of combinations and compositions described herein and the duration of time of efficacy of such combinations, compositions, and radiation therapies. Rest periods of regimens described herein include a period of time in which no agent (e.g., a polypeptide described herein or an anti-cancer agent described herein) is actively administered, and in certain instances, includes time periods where the efficacy of such agents can be minimal. Rest periods of regimens described herein can include a period of time in which no radiation therapy is actively administered. Combination of active administration and rest in regimens described herein can increase the efficacy and/or duration of administration of the combinations described herein.

The term “pharmaceutically acceptable” as used herein refers to physiologically acceptable compounds, agents, or ingredients recognized by a regulatory agency of the Federal or state government, or another governmental agency with authorization for such approval, or and an agent listed in the U.S. Pharmacopeia, European Pharmacopeia or other generally recognized Pharmacopeia for use in animals, and more particularly in humans.

A “pharmaceutically acceptable excipient,” refers to a substance that aids the administration of an active agent to a subject by for example modifying the stability of an active agent or modifying the absorption by a subject upon administration. A pharmaceutically acceptable excipient typically has no significant adverse toxicological effect on the patient. Examples of pharmaceutically acceptable excipients include, for example, water, NaCl (including salt solutions), normal saline solutions, sucrose, glucose, binders, fillers, disintegrants, lubricants, coatings, sweeteners, flavors, alcohols, oils, gelatins, carbohydrates such as amylose or starch, fatty acid esters, hydroxymethycellulose, polyvinyl pyrrolidine, and colors, and the like. One of skill in the art will recognize that other pharmaceutical excipients known in the art are useful in the present invention and include those listed in for example the Handbook of Pharmaceutical Excipients, Rowe R. C., Shesky P. J., and Quinn M. E., 6th Ed., The Pharmaceutical Press, RPS Publishing (2009). The terms binder, filler, disintegrant, and lubricant are used in accordance with the plain and ordinary meaning within the art.

In certain embodiments, a pharmaceutically acceptable excipient may be incompatible (e.g., cross-reacts) with other excipients or active agents described herein. In some embodiments, magnesium stearate, croscarmellose sodium, lactose, excipients comprising Mg, Ca, K, Li, or nucleic acid, acesulfame potassium, ammonium alginate, calcium acetate, calcium alginate, calcium carbonate, calcium chloride, calcium lactate, calcium phosphate, calcium silicate, calcium stearate, calcium sulfate, carboxymethylcellulose calcium, carboxymethylcellulose sodium, docusate sodium, glycine, kaolin, magnesium aluminum silicate, magnesium carbonate, magnesium oxide, magnesium silicate, magnesium trisilicate, polacrilin potassium, polymethacrylates, potassium alginate, potassium benzoate, potassium bicarbonate, potassium chloride, potassium citrate, sodium alginate, sodium benzoate, sodium chloride, sodium lauryl sulfate, sodium starch glycolate, sodium stearyl fumarate, sulfobutylether beta-cyclodextrin, sodium stearate, talc, or zinc stearate are incompatible in the dosage forms described herein.

The term “anti-cancer agent” is used in accordance with its plain ordinary meaning and refers to a composition having anti-neoplastic properties or the ability to inhibit the growth or proliferation of cells. In certain embodiments, an anti-cancer agent is a chemotherapeutic. In certain embodiments, an anti-cancer agent is an agent identified herein having utility in methods of treating cancer. In certain embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” or “cancer therapy” refers to a therapy or regimen that includes administration of a combination, chemotherapeutic, or anti-cancer agent described herein.

The term “radiation therapy” is used in accordance with its plain ordinary meaning and refers to the medical use of radiation in the treatment of cancer. Preferably, the medical use of radiation in the treatment of cancer results in the killing of cancer cells in the subject. A variety of radiation therapies as anti-cancer agents can be used in accordance with the present disclosure, examples of which are provided herein.

A “CD44-modulating polypeptide” refers to a polypeptide that binds to CD44 and modulates its activity (e.g., signaling activity). A CD44-modulating polypeptide can be a polypeptide sequence described herein or, in some embodiments, an antibody that specifically binds to CD44 and inhibits its downstream signaling activity. In one embodiment, a CD44-modulating polypeptide can be a polypeptide sequence described herein or, in some embodiments, an antibody that disrupts or inhibits signaling activity of a CD44 dependent co-receptor. In certain instances, the CD44 dependent co-receptor is a receptor tyrosine kinase (RTK) such as, for example, Met, Ran, or VEGFR. In still another embodiment a CD44-modulating polypeptide can be a polypeptide sequence described herein or, in some embodiments, an antibody that disrupts CD44 co-receptor function or association of a CD44 co-receptor with CD44 or another signaling protein. In one embodiment, a CD44-modulating polypeptide described herein binds to CD44 and inhibits CD44 signaling activity or association with one or more ABC transporters. The ABC transporter can be a multidrug resistant protein (e.g., MDR1). In certain embodiments, CD44 levels can be elevated upon radiation therapy. Exemplary CD44-modulating polypeptides include polypeptides having homology to the CD44-v6 region of human CD44. Such peptides can include substitution variants, addition variants, or chemical derivatives thereof including peptidomimetics.

Provided herein are methods for treating a resistant or refractory cancer, where the cancer can be resistant to at least one anti-cancer agent or radiation therapy by administering a CD44-modulating polypeptide provided herein in combination with an anti-cancer agent described herein. Also provided herein are methods for treating a resistant or refractory cancer, where the cancer can be resistant to at least one anti-cancer agent or radiation therapy by administering a CD44-modulating polypeptide provided herein in combination with radiation therapy described herein. Further provided herein are methods for treating a resistant or refractory cancer, where the cancer can be resistant to at least one anti-cancer agent or radiation therapy by administering a CD44-modulating polypeptide provided herein in combination with an anti-cancer agent described herein and radiation therapy described herein. It is well known in the art that many cancers are resistant or refractory to many anti-cancer agents or radiation therapy(ies) or over the course of treatment, become resistant or refractory to treatment. The methods described herein can restore activity of anti-cancer agents having reduced or eliminated activity against one or more cancers and permit additional treatment options for cancer patients. In another embodiment, the methods described herein can restore activity of radiation therapies described herein having reduced or eliminated activity against one or more cancers and permit additional treatment options for cancer patients.

The present invention includes embodiments where a CD44-modulating polypeptide described herein establishes, restores or enhances the anti-cancer activity of an anti-cancer agent in treating a cancer that is resistant or refractory to the treatment. In one example, a CD44-modulating polypeptide establishes anti-cancer activity (e.g., creates efficacy of an anti-cancer agent in treating a cancer) of an anti-cancer agent described herein in the treatment of cancer. In another example, a CD44-modulating polypeptide restores the anti-cancer activity of an anti-cancer agent described herein. In another example, a CD44-modulating polypeptide enhances the anti-cancer activity of an anti-cancer agent described herein. In still another example, a combination therapy of a CD44-modulating polypeptide described herein and an anti-cancer agent described herein establishes, restores, or enhances activity of a CD44-modulating polypeptide.

The present invention includes embodiments where a CD44-modulating polypeptide described herein establishes, restores or enhances the anti-cancer activity of a radiation therapy in treating a cancer that is resistant or refractory to the treatment. In one example, a CD44-modulating polypeptide establishes anti-cancer activity (e.g., creates efficacy of a radiation therapy) of a radiation therapy described herein in the treatment of cancer. In another example, a CD44-modulating polypeptide restores the anti-cancer activity of a radiation therapy described herein. In another example, a CD44-modulating polypeptide enhances the anti-cancer activity of a radiation therapy described herein. Further provided herein, a CD44-modulating polypeptide can establish, restore, or enhance the anti-cancer activity of one or more anti-cancer agents and radiation therapy. In still another example, a combination therapy of a CD44-modulating polypeptide described herein and a radiation therapy described herein establishes, restores, or enhances activity of a CD44-modulating polypeptide.

The cancer can optionally be resistant or refractory to a plurality of anti-cancer agents (e.g. two or more anti-cancer agents) and/or a plurality of radiation therapies. In one example the cancer can also be resistant, refractory, or non-responsive to treatment with a CD44-modulating polypeptide described herein. In one embodiment of methods of treating described herein, a patient can be administered a combination of a CD44-modulating polypeptide described herein and an anti-cancer agent where the cancer treated is resistant, refractory, or non-responsive to one of or both the CD44-modulating polypeptide and the anti-cancer agent. In one example, the cancer can be resistant, refractory, or non-responsive to treatment with the anti-cancer agent. Administration of the combination of the CD44-modulating polypeptide and anti-cancer agent(s) surprisingly can restore or enhance the activity of the anti-cancer agent against the refractory, resistant, or non-responsive cancer. Administration of the combination of the CD44-modulating polypeptide and anti-cancer agent(s) surprisingly can restore or enhance the activity of the CD44-modulating polypeptide against the refractory, resistant, or non-responsive cancer. Administration of the combination of the CD44-modulating polypeptide and anti-cancer agent(s) surprisingly can restore or enhance the activity of the CD44-modulating polypeptide and the anti-cancer agent against the refractory, resistant, or non-responsive cancer.

In one embodiment of methods of treating described herein, a patient can be administered a combination of a CD44-modulating polypeptide described herein and a radiation therapy where the cancer treated is resistant, refractory, or non-responsive to one of or both the CD44-modulating polypeptide and the radiation therapy. In one example, the cancer can be resistant, refractory, or non-responsive to treatment with radiation therapy. Administration of the combination of the CD44-modulating polypeptide and anti-cancer agent(s) surprisingly can restore or enhance the activity of the radiation therapy against the refractory, resistant, or non-responsive cancer. Administration of the combination of the CD44-modulating polypeptide and anti-cancer agent(s) surprisingly can restore or enhance the activity of the CD44-modulating polypeptide against the refractory, resistant, or non-responsive cancer. Administration of the combination of the CD44-modulating polypeptide and radiation therapy surprisingly can restore or enhance the activity of the CD44-modulating polypeptide and the radiation therapy against the refractory, resistant, or non-responsive cancer.

In one example, a CD44-modulating polypeptide described herein does not have activity against a cancer described herein when administered alone. In one embodiment, where a CD44-modulating polypeptide described herein does not have activity against a cancer described herein when administered alone, its activity can be established or restored when administered in combination with an anti-cancer agent described herein, a radiation therapy described herein, or a combination thereof. In another example, a CD44-modulating polypeptide described herein has minimal activity against a cancer described herein (e.g., insufficient anti-cancer activity to treat a cancer described herein) when administered alone. In one embodiment, where a CD44-modulating polypeptide described herein has minimal activity against a cancer described herein, its activity can be enhanced when administered in combination with an anti-cancer agent described herein, a radiation therapy described herein, or a combination thereof.

In another example, an anti-cancer agent described herein or a radiation therapy described herein does not have activity against a cancer described herein when administered alone (or in combination with another anti-cancer agent). In one embodiment, where an anti-cancer agent described herein or a radiation therapy described herein does not have activity against a cancer described herein when administered alone, its activity can be restored when administered in combination with CD44-modulating polypeptide described herein. In another example, an anti-cancer agent described herein or a radiation therapy described herein has minimal activity against a cancer described herein (e.g., insufficient anti-cancer activity to treat a cancer described herein) when administered alone. In one embodiment, where an anti-cancer agent described herein or a radiation therapy described herein has minimal activity against a cancer described herein, its activity can be enhanced when administered in combination with a CD44-modulating polypeptide described herein. In another example, an anti-cancer agent or a radiation therapy can lose its anti-cancer activity over the course of treatment due to, for example, progression of resistance or refraction in the cancer treated. In one embodiment, the loss of anti-cancer agent or a radiation therapy activity can be slowed, stopped, or reversed (e.g. enhanced activity) when the patient is administered the anti-cancer agent or a radiation therapy in combination with a CD44-modulating polypeptide described herein.

Non-limiting examples of standard methods to measure efflux of drugs include a direct measurement of transport of a fluorescent compound, e.g., Fura-2 (1-[6-amino-2-(5-carboxy-2-oxazolyl)-5-benzofuranyloxy]-2-(2-amino-5-methylphenoxy)ethane-N,N,N′,N′-tetraacetic acid, pentapotassium salt), use of DNA inter-chelating dyes (e.g., Hoeschst H33342, ethidium bromide), an indirect measurement of intracellular accumulation of a substrate, in vivo imaging, and radiolabeled anti-cancer agents. Other examples methods for measuring drug efflux may comprise single-cell or cell-free technologies and/or mass spectrometry.

What is claimed is:

1. A method for preventing efflux of a therapeutic agent that has been internalized into a cell, the method comprises contacting an anti-efflux agent to a region of a protein associated with the cell, wherein the region of the protein in part comprises amino acid sequence having a sequence specified in the PROV.
